(19) United States
(12) Patent Application Publication
(10) Pub. No.: US 2022/0240824 A1
(43) Pub. Date: Aug. 4, 2022

(54) SYSTEM AND METHOD FOR RECOGNISING AND MEASURING AFFECTIVE STATES

(71) Applicant: Tawny GmbH, München (DE)

(72) Inventors: Marco MAIER, München (DE); Michael BARTL, München (DE)

(73) Assignee: Tawny GmbH, München (DE)

(21) Appl. No.: 17/611,588

(22) PCT Filed: May 13, 2020
(86) PCT No.: PCT/EP2020/063393

§ 371 Date: Nov. 16, 2021

(30) Foreign Application Priority Data
May 16, 2019 (DE) 10 2019 113 002.6

(51) Int. Cl. A61B 5/16; G06N 3/08; G06N 3/04

(52) U.S. Cl. CPC A61B 5/165; G06N 3/04; G06N 3/088

(57) ABSTRACT

The affective state called flow is described as a state of optimal experience, total immersion, and high productivity. As an important metric for various scenarios ranging from (professional) sports to work environments and user experience evaluation, it is widely studied using traditional questionnaires. To make flow measurement accessible for online real-time environments, to automatically determine a user's flow state based on physiological signals measured with a wearable device, a system and method is presented to play the game Tetris® at different difficulty levels, resulting in boredom, stress, and flow. A CNN is used to achieve 70% accuracy in detecting flow-inducing values. Disclosed is a training method that has the steps of: Providing an environment configured to place a training subject in a set of affective states, wherein the set of affective states includes at least a first affective state and a second affective state, and the first affective state and the second affective state are different, Providing a system for recognizing affective states, wherein the system is a self-learning system and includes a first input device for inputting physiological information about a training subject and a second input device for inputting the presence of an affective state of the training subject, placing the training subject in an affective state from the set of affective states, acquiring the physiological information about the training subject, determining the affective state, storing the acquired physiological information about the training subject while assigning the determined affective state, inputting the acquired physiological information to the first inputting means, inputting the determined affective state to the second inputting means, and processing the input in the first inputting means and in the second inputting means to train the system to recognize affective states.

The invention relates to a system and method for recognizing affective states of a subject. The invention further relates to a system and a method for setting up a system for recognizing affective states. More particularly, the invention relates to the recognition of affective states using physiological signals.

The research field “Affective Computing” (hereafter “AC”) deals with the recognition, processing, interpretation and simulation of human affects and emotions. With respect to the object of recognizing emotions, typical approaches rely on various kinds of sensor data such as images, videos, audio data, and physiological signals such as heart rate (HR) or electrodermal activity (EDA). Affects detected or recognized in this way can then be used, for example, to control computer systems or a user's environmental variables, such as mood-based adjustment of ambient lighting or temperature.

In addition to basic emotions such as joy, fear, sadness, anger, or surprise, other psychological models such as flow theory can be a valuable construct to assess a user's affective state. The flow state is characterized by optimal experience, full immersion, and high productivity, making it an interesting piece of information when assessing user experience, media use, work productivity, or more generally, states of overload or underload in the context of performing activities.

Traditionally, questionnaires are used to determine whether a subject experiences flow or not, which has the disadvantage that the flow state can only be recorded after it has actually occurred and requires manual work on the part of the subject. Measurement by questionnaires is also subject to the bias of frequently distorted self-assessment and self-evaluation.

It is therefore the object of the invention to provide systems and methods that eliminate the disadvantage from the prior art and provide a way to enable and automate an implicit flow measurement

This object is solved with a method for setting up a system for recognition affective states of a subject according to claim 1, a system for setting up a system for recognizing affective states of a subject according to claim 10, a system for recognizing affective states of a subject according to claim 16, and a method for recognizing affective states of a subject according to claim 17.

Advantageous further developments are the subject-matter of the dependent claims.

Disclosed is a method of setting up a system for recognizing affective states of a subject, comprising the steps of: a. providing an environment configured to place a training subject in a set of affective states, wherein the set of affective states includes at least a first affective state and a second affective state, and the first affective state and the second affective state are different; b. providing a system for setting up a system for recognizing affective states, wherein the system is a learning system and includes a first input device for inputting physiological information about a training subject, and a second input device for inputting or automatically detecting physiological information about a training subject, c. putting the training subject into an affective state from the set of affective states, d. acquiring the physiological information about the training subject, e. determining the affective state, f. storing the acquired physiological information about the training subject while assigning the determined affective state, g. inputting the acquired physiological information into the second input device, and h. inputting the determined affective state into the second input device to train the system for recognizing affective states. Here, the input via the first input device and the second input device may also be performed by providing the input via a unified data block, for example as feeding a file to the system, wherein the respective data are separated in an internal process and then subjected to different processing operations. Furthermore, in this case, putting the training subject into an affective state does not necessarily have to be done by the system or the process itself. Here, an external stimulus or the affective state brought along by the training subject is also sufficient. All that is required is that the training subject is in a defined affective state during training. Furthermore, the set of at least two affective states can also consist of an affective state and its complement, for example, anxious or anxiety-free or stressed or stress-free.

Disclosed is the method of setting up a system for recognizing affective states of a subject, wherein steps c. to i. are repeated for multiple training subjects or for multiple affective states.

Disclosed is the method of setting up a system for recognizing affective states of a subject, wherein the set of affective states includes the affective state boredom/underchallenge, the affective state flow, and the affective state stress/overload.

Disclosed is the method of setting up a system for recognizing affective states of a subject, wherein the physiological information is visual information, physiological signal information, or acoustic information.

Disclosed is the method of setting up a system for recognizing affective states of a subject, wherein the visual information is still image information or moving image information of a human face, the physiological signal information is electrodermal activity information, heart rate information, and heart rate variance information, or the acoustic information is a recording of a human voice.

Disclosed is the method of setting up a system for recognizing affective states of a subject, wherein the environment configured to put the training subject into a set of affective states comprises a task setting device, for example an electronic data processing device, by means of which the training subject is set a task, for example to play the game Tetris®, wherein the task is configured to have at least as many difficulty levels as there are affective states in the set of affective states, and there is an objective assignment of difficulty levels to the affective states in the form that the training subject is put into a certain affective state when solving the task in one of the difficulty levels.

Disclosed is the method of setting up a system for recognizing affective states of a subject, wherein the game Tetris® has difficulty levels of easy, medium, and hard, and the difficulty levels are configured such as to place the training subject in the state of boredom, flow, and stress, respectively, when playing the respective difficulty level.

Disclosed is the method of setting up a system for recognizing affective states of a subject, wherein the learning system comprises a neural network, a convolutional neural network (CNN), or a recurrent neural network (RNN).

Disclosed is the method of setting up a system for recognizing affective states of a subject, wherein the learning system comprises a convolutional neural network consisting of four convolutional layers with 32 filters and a kernel size of 3, the layers being connected via max-pooling layers, after the convolutions, a fully connected layer with 32 neurons leads to a final dense layer, and the final dense layer has a number of neurons corresponding to the number of classes of the classification task and a Softmax activation.

Disclosed is a system for establishing a system for recognizing affective states of a subject, comprising: an environment configured to place a training subject in a set of affective states, the set of affective states including at least a first affective state and a second affective state, the first affective state and the second affective state being different, a first input device for inputting physiological information about a training subject and a second input device for inputting the presence of an affective state of the training subject, wherein the system is a learning system and is configured and determined to perform the above method of setting up a system for recognizing affective states of a subject.

Disclosed is the system for setting up a system for recognizing affective states of a subject, wherein the environment arranged to put the training subject into a set of affective states comprises an electronic data processing device by means of which the training subject is arranged to play the game Tetris®, the game being arranged as many difficulty levels as there are affective states in the set of affective states, and there is an objective assignment of difficulty levels to the affective states in the form that the training subject is put into a certain affective state when playing the game Tetris® in one of the difficulty levels.

Disclosed is the system for setting up a system for recognizing affective states of a subject, the game Tetris® having difficulty levels of easy, medium, and hard, the difficulty levels being arranged to place the training subject in the state of boredom, flow, and stress, respectively, when playing the respective difficulty level.

Disclosed is the system for setting up a system for recognizing affective states of a subject, wherein the learning system comprises a neural network, a convolutional neural network (CNN), or a recurrent neural network (RNN).

Disclosed is the system for setting up a system for recognizing affective states of a subject, the learning system comprising a convolutional neural network consisting of four convolutional layers with 32 filters and a kernel size of 3, the layers being connected via max-pooling layers, after the convolutions, a fully connected layer with 32 neurons leads to a final dense layer, and the final dense layer has a number of neurons corresponding to the number of classes of the classification task and comprising a Softmax activation.

Disclosed is the system for setting up a system for recognizing affective states of a subject, wherein the first input device for inputting physiological information about a training subject is connected to a camera for capturing moving image information of a face of the training subject, a wristband device that detects physiological signals such as electrodermal activity, heart rate, or heart rate variability, or a microphone for detecting a voice of the training subject.

Disclosed is a system for recognizing affective states of a subject, wherein the system is learning and has been trained according to the method for setting up a system for recognizing affective states of a subject according to any of the preceding paragraphs.

Disclosed is a method for recognizing affective states of a subject, comprising the steps of: a. providing a system according to the preceding claim, comprising a first input device for inputting physiological information about a subject and a first output device for outputting the presence of an affective state of the subject, b. acquiring and inputting the physiological information about the subject to the first input device, c. processing the input of the physiological information by the system, d. classifying an affective state of the subject by the system, e. outputting the classified affective state of the subject via the first output.

Disclosed is the method of recognizing affective states of a subject according to any of the preceding paragraphs, wherein the classified affective state of the subject is one of boredom, flow, and stress.

Disclosed is the method of recognizing affective states of a subject according to any of the preceding paragraphs, wherein the physiological information is visual information, physiological signal information, or acoustic information.

Disclosed is the method for recognizing affective states of a subject according to the preceding paragraph, wherein the visual information is moving image information of a human face, the physiological signal information is electrodermal activity information, heart rate information, and heart rate variance information, or the acoustic information is a recording of a human voice.

Disclosed is a system for recognizing affective states of a subject that configured to perform any of the preceding methods for recognizing affective states of a subject, comprising: the system for recognizing affective states of a subject according to any of the preceding paragraphs, the first input device for inputting physiological information about the subject, and a first output device for outputting the presence of an affective state of the subject.

Disclosed is a system for recognizing affective states of a subject according to any one of the preceding paragraphs, wherein the first input device comprises a camera for capturing moving images, a wristband device that detects physiological signals such as electrodermal activity, heart rate, or heart rate variability, or a microphone for voice recording.

Disclosed is the system for recognizing affective states of a subject according to any of the preceding paragraphs, comprising a central service, wherein the central service is divided into at least the following layers: an interaction layer arranged to allow users to transmit physiological information to the system for recognizing affective states of the subject, an evaluation layer arranged to determine the affective state of the subject from the transmitted physiological information by means of learning system trained by the method for setting up a system for recognizing affective states of a subject according to any one of the preceding claims, and a database layer storing training data for training the learning system.

Disclosed is the system for recognizing affective states of a subject according to any of the preceding paragraphs, wherein the interaction layer provides the user with a user interface accessible over a wide area network, through which files of physiological information can be transmitted to the system, and through which information about the affective state associated from the physiological information is displayed to the user or provided to the user as a transmittable file.

Disclosed is the system for recognizing affective states of a subject according to any of the preceding paragraphs, wherein the system is configured to provide the interaction layer to an investigative user by means of a user interface accessible via the wide area network, through which the investigative user can provide content consumed by a target user, the interaction layer is configured to enable the target user to communicate physiological information captured during consumption to the system, and configured to display to the investigative user, or provide to the investigative user as a transferable file, information about the affective state associated with the physiological information.

Disclosed is the system for recognizing affective states of a subject according to one of the preceding paragraphs, wherein the evaluation layer is divided into several modules, a visual module is configured to determine the affective state of the subject from visual information, in particular moving image information of a human face as physiological information, a physiological module is set up to determine the affective state of the subject from physiological signal information, in particular electrodermal activity information, heart rate information and heart rate variance information as physiological information, and an acoustic module is set up to determine the affective state of the subject from acoustic information, in particular the recording of a human voice as physiological information.

Abstract: The affective state called “flow” is described as a state of optimal experience, total immersion and high productivity. As an important metric for various scenarios ranging from (professional) sports to work environments to user experience evaluations, conventionally it is extensively studied using traditional questionnaires. In order to make flow measurement accessible for online, real-time environments, this invention present findings towards automatically estimating a user's flow state based on physiological signals measured with a wearable device or captured via image recording (moving or still). Here, a study is conducted of subjects playing the game Tetris® in varying difficulty levels, leading to boredom, stress, and flow. Using a convolutional neural network, an accuracy of 70% is achieved in recognizing flow-inducing levels. As a possible future development, flow is expected to be a potential reward signal for human-in-the-loop reinforcement learning systems.

Introduction: The research field “Affective Computing” (hereafter “AC”) is dealing with recognizing, processing, interpreting, and simulating human affects and emotions. With regard to the goal of recognizing emotions, typical approaches rely on various kinds of sensor data like images, videos, audio data], and physiological signals such as heart rate (HR) or electrodermal activity (EDA). Besides basic emotions such as joy, fear, sadness, anger or surprise, other psychological models such as the flow theory can be a valuable construct to assess a user's affective state. The state of flow is characterized by optimal experience, total immersion and high productivity, making it an interesting piece of information when assessing user experiences, from user interfaces to games to whole environments. Traditionally, whether a subject experiences flow or not is determined through questionnaires, which has the disadvantage of being only applicable after the actual occurrence and requires manual effort from the subject. In contrast, automatic flow recognition based on sensor data would be applicable implicitly, unobtrusively and in real-time.

The invention discloses a method for automatically measuring flow with physiological signals from, for example, wrist-worn devices. The method is based on a CNN (Convolutional Neural Network) architecture. A study setup using the well-known game Tetris® is presented for the generation of training data. Results are revealed using a pilot study.

Study Design: For the data collection, we created a custom version of the game Tetris® as a mobile application. Tetris has already been used in similar studies and it has been found that depending on the difficulty of the game, users experience flow. The original game logic was modified so that there are only three different levels, i.e., easy, normal, and hard, in random order, each lasting 10 minutes, and independent from the player's performance. The difficulty of the three levels, i.e., the speed of the falling tetriminos, was set how it was expected in advance that the game to lead to boredom, flow, and stress respectively . . . .

Participants were selected so that they all had approximately the same skill level in the game. They were equipped with an Empatica E4 wrist-worn device capturing physiological signals such as electrodermal activity (hereafter EDA), heart rate (hereafter HR), and heart rate variability (hereafter HRV). The E4 was worn on the participant's non-dominant hand. The smartphone (iPhone 5s) with the Tetris® application was held in the other (dominant) hand.

The following preliminary evaluation were conducted on a data set from a small pilot study. There were eleven participants (three female, eight male) between the ages of 20 and 35. In total, we gathered 31 sessions, summing up to 15.5 hours of data. 4 participants played several sessions, 7 played only one session.

Data and preprocessing: Three streams of physiological signals were used from the E4: HR, HRV, and EDA. HR and EDA are provided by the E4 and were used in its raw form. With regard to HRV, the E4 provides the so-called RR-intervals, i.e., the time difference between consecutive heart beats, from which various HRV measures can be derived. EDA is sampled at 4 Hz while the HR values are provided at 1 Hz. RR intervals are not provided at regular intervals but when they occur. In order to align the RR intervals with the two other data streams, a common HRV measure was calculated called RMSSD (root mean square of successive differences) [18].

TABLE 1
Best mean test accuracies achieved in leave-one-session-out and
leave-one subject-out cross validation.

| | | Leave-one- | Leave-one- |
|---|---|---|---|
| Accuracy (%) | Baseline | session-out | subject-out |
| Boredom vs. not boredom | 50.00 | 65.04 | 57.13 |
| Flow vs. non-flow | 50.00 | 70.37 | 69.55 |
| Stress vs. non-stress | 50.00 | 66.09 | 71.17 |
| Boredom vs. flow vs. stress | 33.33 | 52.59 | 50.43 |

The RMSSD measure is computed over windows of data and it is recommended to use a window size of at least 60 seconds. Consequently, at each time step where an RR value was received, a window of size 60 seconds before this point in time was extracted and the RMSSD value was computed for that window. The sample times of the EDA series were used as a basis for the final time series. Both HR and RMSSD values were forward-filled to fit the 4 Hz sampling frequency of the EDA series. The result is an equidistant time series, sampled at 4 Hz, with EDA, HR and HRV (i.e., RMSSD) values at each time step.

To create the training and validation sets, each session was divided into ranges of n samples. In order to create the training and validation sets, we split each session in windows of n samples. The window interval slides forward one sample at a time, i.e., consecutive windows overlap by n 1 samples. For this work, we used 10 second windows, i.e., windows consisting of n=40 samples, each containing three values. The window length of 10 seconds was chosen because preliminary tests showed that shorter windows do not allow to capture characteristic patterns.

The approach is based on a convolutional neural network architecture. The network consists of four convolutional layers (32 filters, kernel size 3), connected through max pooling layers. After the convolutions, one fully connected layer (32 neurons) leads to a final dense layer with the number of neurons in accordance with the number of classes of the classification task and a Softmax activation. Except for the last layer, we used ReLU activations for the layers. During training, dropout is applied after the convolutional (0.1) and dense (0.5) layers to prevent overfitting

Three binary one-vs-all classification tasks were evaluated and one task trying to distinguish between all three classes at the same time. When creating the training and validation data sets, examples were chosen in a balanced manner, i.e., for the binary tasks, only half of the examples for the negative class were randomly drawn from the available examples to keep an even split between the two classes. We trained and evaluated our model in two ways: leave-one-session-out cross validation and leave-one-subject-out cross validation, the latter only on subjects that had played only one session, thus, validating on a completely unseen subject in each iteration. Table 1 shows the results.

It can be seen that the examples associated with boredom are the hardest to get right. It is supposed that the easy level leads to the highest diversity of feelings among the three levels, i.e., the very slow speed is sometimes perceived as relaxing, sometimes as stressful, and only sometimes as distinctively boring. All in all, the CNN model is able to differentiate between the three classes considerably more accurately than the baseline strategy.

As outlined before, the affective state of flow is often associated with high productivity or better performance. In the case at hand, the achieved score in the Tetris® game can be interpreted as the user's productivity. Thus, the model can be applied to Tetris® sessions in order to divide a session into intervals of boredom, flow and stress—this time without taking into account information about the actual game level—and then observe how good the performance of the player is in the respective states. Players indeed performed best when the model has recognized the flow state (average of 2.59 points per 10-second window), second best when the player is estimated to be bored (2.04 points). In contrast, when our system recognizes the state of stress, players perform considerably worse, even obtaining negative scores during these phases (−0.50 points).

From a psychological perspective, it should be further verified if the affective states we are trying to induce with the different difficulty levels of the game really can be considered boredom, stress and flow. Even though our general setup is in accordance with previous studies examining flow and especially examining flow with the game Tetris, the exact variant of the game and the surrounding conditions have not been fully verified. Thus, combining our data collection process with psychology-validated flow questionnaires is advisable.

On the other hand, we could observe that players perform best during time intervals our model classifies as flow, which could be regarded as an indicator for an actual flow experience.

All in all, the positive initial results open up several possibilities for further work. In addition to improving the data set and adapting the model, there is much potential in transferring the general approach to other similar tasks, especially typical tasks of an office job.

More clearly scoped to the field of AI research, it is especially interesting to use automatic flow detection as a feedback mechanism in human-in-the-loop reinforcement learning. Socially intelligent agents could benefit from the information about this affective state by incorporating it as a reward signal for their behavior.

Disclosed is a central service with multiple layers. The top layer represents an interaction layer. Users can upload video, audio or biometric data and receive the emotion analysis of their content back in an online dashboard or optionally as a raw data download (e.g. csv file) for individual further processing. Alternatively, users can invite other participants to test applications in real time via the platform. Facial emotions, speech, wearable data are recorded while consuming digital content (e.g. video, advertisement, product presentation, etc.) or performing a test task (e.g. app, website UX/UI, interview, chat bot, game, etc.).

The middle layer is an evaluation layer. The platform is designed to preprocess human data such as facial expressions, speech or biometric data from wearable sensors such as heart rate variability (HRV), heart rate (HR), skin electrodermal activity (EDA), skin temperature, photoplethysmography (blood volume pulse) and motion (accelerometer, gyroscope).

Processing can be performed individually or in various combinations. Neural networks and deep learning techniques are embedded in the distributed and real-time software architecture to identify patterns for classifying human states such as emotional stress, happiness, attention, productivity level, flow, etc. Classification of human affects is supported by inventories, scales, and tests known from psychophysiology and social science (e.g., PANAS scale, Oxford happiness catalog, flow construct, etc.). A visual module, a physiological module and an acoustic module are the out-of-the-box core modules, which can additionally be offered for integration into other (software) products and customized business solutions on a royalty basis.

The base layer is a database layer used to train machine learning models such as Convolutional Neural Networks (CNN) and Recurrent Neural Networks (RNN) to classify human emotional conditions based on visual, auditory, and physiological data. This proprietary data enabled the creation of a learning environment for emotional classification models.

The invention is explained below with reference to an example of an embodiment and the figures.

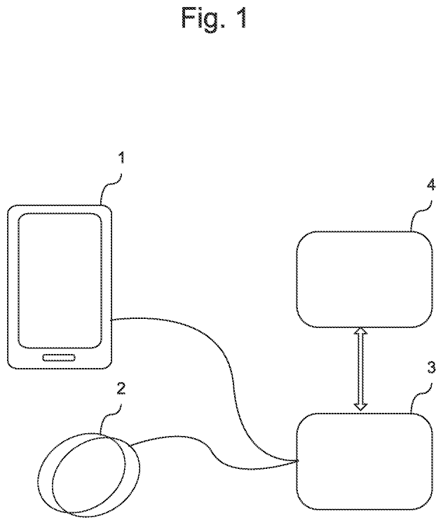

FIG. 1 exhibits a system according to the invention for setting up a system for recognizing affective states of a subject.

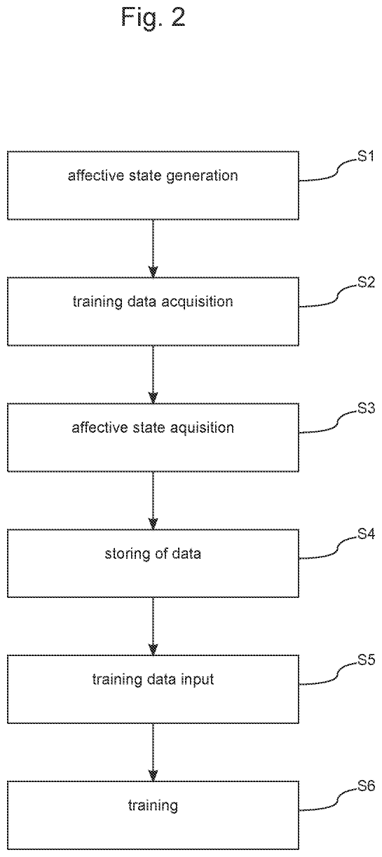

FIG. 2 exhibits a flowchart of a method according to the invention for setting up a system for recognizing affective states of a subject.

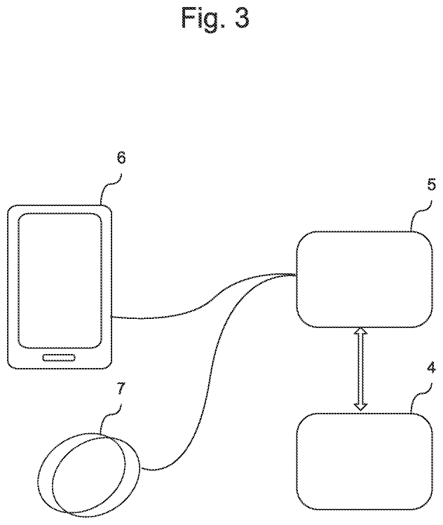

FIG. 3 exhibits a system according to the invention for recognizing affective states of a subject.

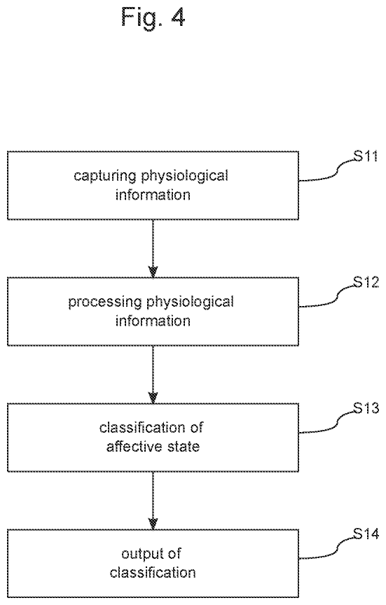

FIG. 4 exhibits a flowchart of a method according to the invention for recognizing affective states of a subject.

With reference to FIG. 1, a system according to the invention for setting up a system for recognizing affective states of a subject is described.

The device system has an affective state generation device 1. In the embodiment example, the affect state generation device 1 is an electronic data processing device, in particular a mobile data processing device such as a smartphone or a tablet, on which a modified form of the computer game Tetris® is installed. The game Tetris® is modified to have three difficulty levels of easy, normal, and difficult. The difficulty levels are designed so that an average player experiences the affective state of “boredom” when playing Tetris® at the easy difficulty level, the affective state of “flow” at the medium difficulty level, and the affective state of “stress” at the difficult difficulty level.

The experimental setup with the modified form of the computer game Tetris® is here only one possible trigger of an affective state. Other stimulants are also conceivable, such as typing, Hangman, solving crossword puzzles, taking intelligence tests or aptitude/screening tests such as the aptitude test for applying for medical university education.

The set-up system has a body information acquisition device 2. The body information acquisition device 2 is a device for acquiring physiological information about the training subject. In the embodiment example, the body information acquisition device 2 is an Empatica E4® wristband device that acquires physiological signals such as electrodermal activity, heart rate, and heart rate variance.

The device system further comprises a training data storage device 3. The training data storage device 3 is an electronic data processing device connected to the affective state generation device 1 and the body information acquisition device 2 by means of electronic data communication, for example Bluetooth or WLAN, such that information can be transmitted from the affect state generation device 2 and the body information acquisition device 2 to the training data storage device 3.

The setup system further comprises a training device 4. The training device 4 is an electronic data processing device implementing a Convolutional Neural Network (CNN) architecture. The CNN consists of four convolutional layers (32 filters, kernel size 3) connected by max-pooling layers. After the convolutions, a fully connected layer (32 neurons) leads to a final dense layer (dense layer) with the number of neurons corresponding to the number of classes of the classification task and a Softmax activation. The training device 4 is connected to the training data storage device 3 by means of electronic data communication, for example Ethernet, such that training data can be transmitted from the training data storage device 3 to the training device 4.

With reference to FIG. 2, a method for setting up a system for recognizing affective states of a subject is explained.

In an affect state generation step S1, a training subject is put into a defined affective state. For this purpose, the training subject is given the task of playing the modified game Tetris® at a predefined difficulty level using the affect state generation device 1.

During the game, in a training data acquisition step S2, the physiological information of electrodermal activity, heart rate, and heart rate variance is acquired by the body information acquisition device 2 and transmitted to the training data storage device 3.

In a state detection step S3, the affective state associated with the difficulty level is detected and transmitted to the training data storage device 3.

In a training data storage step S4, the physiological information determined in the training data acquisition step S2 is stored in the training data storage device 3 by assigning the affective state determined in the state determination step S3.

Steps S1 to S4 can be repeated for different training subjects or different affective states.

In a training data input step S5, the physiological information stored in the training data storage device 3 and the affective states associated with it are supplied to the CNN of the training device 4.

In a training step S6, the CNN of the training device 4 is trained using the physiological information and the affective states associated with them.

With reference to FIG. 3, a system for recognizing affective states of a subject is explained.

The recognition system features the training device 4 of the setup system trained according to the procedure of FIG. 2.

The recognition system further comprises an interaction device 5. The interaction device 5 is an electronic data processing device and provides a user interface by that a user can transmit physiological information to the interaction device 5. For example, a user may transmit a video file of a subject's face or an audio file of a subject's voice or other physiological information such as that of a fitness tracker. The interaction device 5 is connected to the training device 4 such that the physiological information can be transmitted from the interaction device 5 to the training device 4, and correlating affective states can be determined by the training device 4 and transmitted to the interaction device 5. The interaction device 5 is arranged to display or otherwise communicate information about the determined affective state to the user, for example by transmitting a file.

Alternatively, the interaction device 5 is arranged to present content or tasks to a user to be consumed or performed. Here, physiological information of the user as subject during the consumption or execution can be recorded and evaluated by the training device 4.

The training device 4 is configured to have multiple modules. A visual module is configured to classify affective states from visual information, in particular moving image information of a human face. A physiological module is configured to classify affective states from physiological signal information, in particular electrodermal activity information, heart rate information, and heart rate variance information. An acoustic module is set up to determine affective states from acoustic information, in particular the recording of a human voice as physiological information.

The interaction device 5 is accessible to users via a wide area network, for example the Internet. Physiological information can be captured by the user, for example, by sensors of his terminal device, and transmitted to the interaction device 5. For example, on a smartphone 6, the user can transmit a moving image of his face to the interaction device 5 via the camera of the smartphone when solving a task set for him. This moving image enables the determination of heart rate, heart rate variance, and electrodermal activity via skin discoloration. This information is analyzable by the visual module of the training device 4.

With reference to FIG. 4, a method for recognizing affective states of a subject is explained.

In a capturing step S11, physiological information of the subject is captured. This can be done, for example, by capturing a moving image of the subject's face or by capturing immediate physiological signal information such as electrodermal activity, heart rate, and heart rate variance using an Empatica E4 wristband device. The physiological information is obtained by the interaction device 5 and transmitted to the training device 4.

In a processing step S12, the training device 4 processes the physiological information.

In a classification step S13, the training device 4 classifies an affective state associated with the physiological information.

In an output step S14, the classified affective state is transmitted from the training device 4 to the interaction device 5 and displayed by the interaction device 5 to the user in the user interface or offered for transmission, for example as a file.

The invention is not limited to the embodiments described above. Obviously for the person skilled in the art, modifications of the embodiments are possible without falling outside the scope of protection of the invention as defined by the claims.

For example, in the embodiment, the affective state generation device 2 is an electronic data processing device with a modified version of the game Tetris®. Alternatively, the affect state generation device 2 may have other games or applications installed thereon that can place a training subject in an affective state that is communicable to a data storage device.

Alternatively, any other device or environment may be used as an affect state generation device that is capable of placing a training subject in an affective state that is at least indirectly communicable to a data storage device.

In the embodiment example, the body information acquisition device 2 is an Empatica E4® wristband device. Alternatively, any other device capable of capturing and transmitting physiological information of a training subject may be used. In particular, this may be a camera capable of capturing and transmitting moving image information of a face of the training subject.

In the embodiment example, the training data storage device 3, the training device 4 and the interaction device 5 are separate devices. Alternatively, however, the functions of these devices can each be distributed among several devices or combined in different combinations in one device.

### Definitions

The terms listed in the following are used in the description and claims as defined herein:
Subject human participant whose affective state is or ought to be the subject of a state determination.
Training subject human participant whose affective state is the subject of the training of a self-learning system.
Softmax activation a specialized activation function for classification networks with one-of-N coding. It creates a normalized exponential, i.e., the outputs sum to 1. In combination with the cross-entropy error function, it enabled a modification of multilayer perceptron networks to estimate class probabilities.
Physiological information: information concerning one or more measurable properties of a subject's or training subject's body, such as heart rate and heart rate variance. For the purposes of this doctrine, voice modulation, facial expressions, posture, and movement patterns also constitute such physiological information.
Affective state a state of a subject allocated to an emotion or a state of mind, which can be assigned in the valence/emotion space with the two axes valence and arousal and is reflected in physiological information about the subject.
Boredom an affective state of uncomfortable, unpleasant feeling that is caused by forced idleness or can arise during an activity that is perceived as monotonous or underwhelming.
Flow also called functional pleasure or activity inebriety, is an affective state experienced as pleasurable, a feeling of complete immersion (concentration) and complete absorption in an activity, which manifests itself in above-average productivity.
Stress an affective state caused by specific external stimuli (stressors) that leads to physical or mental overload.
[electronic] data processing device:
device that is configured to transmit (sending and receiving), store or process data in electronic form.
and junctor in the meaning of the logical conjunction (mathematical AND).
or junctor in the meaning of logical adjunction (mathematical OR, often also “and/or”).
either . . . or junctor in the meaning of logical contravalence (mathematical exclusive OR).

1. A method of setting up a system for recognizing affective states of a subject, comprising the steps of:
a. providing an environment configured to observe a training subject and/or place the training subject in a set of affective states, wherein the set of affective states comprises at least a first affective state and a second affective state, and the first affective state and the second affective state are different,
b. providing a system (1, 2, 3, 4) for setting up an affective state recognition system, the system being a self-learning system and comprising
a first input device for inputting physiological information about a training subject and
a second input device for inputting or automatically recognizing the presence of an affective state of the training subject,
c. putting the training subject into an affective state from the set of affective states,
d. acquiring the physiological information about the training subject,
e. determining the affective state,
f. storing the acquired physiological information about the training subject with assignment of the determined affective state,
g. inputting the sensed physiological information into the first input device,
h. inputting the determined affective state to the second input device, and
i. processing the input in the first input device and in the second input device to train the system for recognizing affective states.

2. The method of setting up a system for recognizing affective states of a subject according to claim 1, wherein steps c. to i. are repeated for multiple training subjects or for multiple affective states.

3. The method of setting up a system for recognizing affective states of a subject according to claim 1, wherein the set of affective states comprises the affective state boredom, the affective state flow, and the affective state stress.

4. The method of setting up a system for recognizing affective states of a subject according to claim 1, wherein the physiological information is visual information, physiological signal information, or acoustic information.

5. The method of setting up a system for recognizing affective states of a subject according to claim 4, wherein
the visual information is still image information or moving image information of a human face,
the physiological signal information is electrodermal activity information, heart rate information, and heart rate variance information, or
the acoustic information is the recording of a human voice.

6. The method of setting up a system for recognizing affective states of a subject according to claim 1, wherein the environment configured to put the training subject into a set of affective states comprises a task setting device, for example an electronic data processing device (1), by means of which the training subject is set a task, for example to play the game Tetris®, wherein the task is arranged to have at least as many difficulty levels as there are affective states in the set of affective states, and there is a surjective, preferably bijective, assignment of difficulty levels to the affective states in the form that the training subject is put into a certain affective state when solving the task in one of the difficulty levels.

7. The method of setting up a system for recognizing affective states of a subject according to claim 6, wherein the game Tetris® has difficulty levels of easy, medium, and hard, the difficulty levels being arranged to place the training subject in the state of boredom, flow, and stress, respectively, when playing at the respective difficulty level.

8. The method of setting up a system for recognizing affective states of a subject according to claim 1, wherein the learning system comprises a neural network, a convolutional neural network (CNN), or a recurrent neural network (RNN).

9. The method of setting up a system for recognizing affective states of a subject according to claim 8, wherein
the self-learning system comprises a convolutional neural network consisting of four convolutional layers with 32 filters and a kernel size of 3,
the layers being connected via max-pooling layers,
after the convolutions, a fully connected layer with 32 neurons leads to a final dense layer, and
the final dense layer has a number of neurons corresponding to the number of classes of the classification task and comprises a Softmax activation.

10. A system (1, 2, 3, 4) for setting up a system for recognizing affective states of a subject, comprising:
an environment configured to place a training subject in a set of affective states, the set of affective states including at least a first affective state and a second affective state, the first affective state and the second affective state being different,
a first input device for inputting physiological information about a training subject and a second input device for inputting the presence of an affective state of the training subject, wherein
the system is a learning system and
is arranged and determined to perform the method of setting up a system for recognizing affective states of a subject according to claim 1.

11. The system for setting up a system for recognizing affective states of a subject according to claim 10, wherein the environment configured to put the training subject into a set of affective states comprises an electronic data processing device (1) by means of which the training subject is arranged to play the game Tetris®, the game being configured such as to comprise as many difficulty levels as there are affective states in the set of affective states, and there is a bijective assignment of difficulty levels to the affective states in such a way that the training subject is put into a certain affective state when playing the game Tetris® in one of the difficulty levels.

12. The system for setting up a system for recognizing affective states of a subject according to claim 1, wherein the game Tetris® has difficulty levels of easy, medium, and hard, wherein the difficulty levels are configured to place the training subject in the state of boredom, flow, and stress, respectively, when playing the respective difficulty level.

13. The system for setting up a system for recognizing affective states of a subject according to claim 1, wherein the self-learning system (4) comprises a neural network, a convolutional neural network (CNN), or a recurrent neural network (RNN).

14. The system for setting up a system for recognizing affective states of a subject according to claim 13, wherein
the self-learning system (4) comprises a convolutional neural network consisting of four convolutional layers with 32 filters and a kernel size of 3,
the layers are connected via max-pooling layers,
after the convolutions, a fully connected layer with 32 neurons leads to a final dense layer, and
the final dense layer has a number of neurons corresponding to the number of classes of the classification task and comprises a Softmax activation.

15. The system for setting up a system for recognizing affective states of a subject according to claim 1, wherein the first input device for inputting physiological information about a training subject is connected to a camera for capturing moving image information of a face of the training subject, a wristband device (2) that detects physiological signals such as electrodermal activity, heart rate, or heart rate variability, or a microphone for detecting a voice of the training subject.
